(19) United States
(12) Patent Application Publication
(10) Pub. No.: US 2025/0206683 A1
(43) Pub. Date: Jun. 26, 2025

(54) REACTOR FOR NON-OXIDATIVE DIRECT CONVERSION OF METHANE, AND METHOD FOR PREPARING C2 HYDROCARBON COMPOUNDS AND AROMATIC COMPOUNDS BY USING SAME

(71) Applicant: KOREA RESEARCH INSTITUTE OF CHEMICAL TECHNOLOGY, Daejeon (KR)

(72) Inventors: Yong Tae KIM, Daejeon (KR); Jin Ju LEE, Daejeon (KR); Sung Woo LEE, Daejeon (KR); Jung Ho SHIN, Daejeon (KR); Seung Ju HAN, Daejeon (KR); Tsegay Gebrekidan GEBREYOHANNES, Daejeon (KR); Eun Hae SIM, Daejeon (KR)

(73) Assignee: KOREA RESEARCH INSTITUTE OF CHEMICAL TECHNOLOGY, Daejeon (KR)

(21) Appl. No.: 19/076,564

(22) Filed: Mar. 11, 2025

Related U.S. Application Data
(63) Continuation of application No. PCT/KR2023/013323, filed on Sep. 6, 2023.

(30) Foreign Application Priority Data
Sep. 16, 2022 (KR) 10-2022-0116903

(51) Int. Cl. C07C 2/58; B01J 8/00; C07C 9/06; C07C 11/04; C07C 11/24

(52) U.S. Cl. CPC C07C 2/58; B01J 8/008; C07C 9/06; C07C 11/04; C07C 11/24

(57) ABSTRACT

The present invention relates to a non-oxidative direct conversion reactor for methane and a method for producing C2 hydrocarbon compounds and aromatic compounds using the same. More specifically, it concerns a reactor and method that enable the production of C2 hydrocarbon compounds and aromatic compounds from methane, significantly improving methane consumption while ensuring high yields of C2 hydrocarbon compounds and aromatic compounds even at low reaction temperatures.

### CROSS REFERENCE TO RELATED APPLICATION

The present application is a continuation of PCT International Application No. PCT/KR2023/013323, which has an International filing date of Sep. 6, 2023, which claims priority based on Korean Patent Application No. 10-2022-0116903, filed on Sep. 16 2022, the entire contents of each of which are incorporated herein for all purposes by this reference.

### TECHNICAL FIELD

The present invention relates to a methane non-oxidative direct conversion reactor and a method for producing C2 hydrocarbon compounds and aromatic compounds using the same. More specifically, it relates to a methane non-oxidative direct conversion reactor capable of directly converting methane under anaerobic or oxygen-free conditions to produce C2 hydrocarbon compounds and aromatic compounds, and a method for producing C2 hydrocarbon compounds and aromatic compounds using this reactor.

### BACKGROUND ART

In recent years, consistent efforts have been made to convert methane (CH₄), which can be obtained from natural gas and shale gas, into high-value products such as transportation fuel or chemical raw materials. Typical examples of high-value products that can be obtained from methane include olefins (ethylene, propylene, butylene, etc.) and aromatic compounds. MTO (Methanol to Olefins) technology that manufactures light olefins via methanol derived from syngas (H₂+CO) produced through methane reforming, and FTO (Fischer-Tropsch to Olefins) technology, which directly produces light olefins from syngas, are known as the most feasible technologies.

However, in the case of technologies that produce high value-added products via synthesis gas, additional hydrogen (H₂) or carbon monoxide (CO) is required to remove oxygen atoms from carbon monoxide (CO). This results in a decrease in the utilization efficiency of hydrogen atoms or carbon atoms in the overall process.

Therefore, new technologies are required that can convert methane directly into high value-added products without going through synthesis gas. To convert methane directly into high value-added products, it is necessary to first activate methane by breaking the strong C—H bonds (434 KJ/mol) of carbon. From this perspective, research on Oxidative Coupling of Methane (OCM) technology, which activates methane using oxygen, has been actively conducted. However, even in OCM reactions, due to the violent reactivity of O₂, the formation of large amounts of thermodynamically stable H₂O and CO₂ is still pointed out as a problem, resulting in decreased utilization efficiency of hydrogen atoms or carbon atoms.

To address these issues, recent technologies have been developed to produce ethylene, aromatic compounds, and others through the direct conversion of methane under anaerobic or anoxic conditions. However, due to the low reactivity of methane, these processes are carried out at high temperatures and pressures, and the development of suitable reactors and catalysts is essential. According to research results, a critical issue has emerged regarding the rapid decline in catalyst activity caused by carbon (coke) deposition on the catalyst under high-temperature and high-pressure conditions (refer to Non-Patent Literature 0001 and 0002).

In U.S. Pat. No. 4,424,401, a method is disclosed for aromatizing a hydrocarbon mixture by diluting acetylene with inert gases, water, hydrogen, methane, and alcohol in the presence of a zeolite catalyst ZSM-5. In U.S. Pat. No. 8,013,196, a method is disclosed for producing ethylene by thermally converting a feedstock containing methane into an acetylene-containing effluent through pyrolysis and then hydrogenating the converted acetylene-containing effluent.

However, as in these prior references, the aromatization methods of methane or acetylene on zeolite or other catalysts also exhibited issues where the catalyst performance was demonstrated only for a very short period and rapidly deactivated due to the accumulation of coke fragments and the rapid polymerization of acetylene. Furthermore, a high content of other byproducts derived from acetylene conversion was formed.

In particular, the known methods for producing C2 hydrocarbon compounds, aromatic compounds, and others from methane-containing feedstocks exhibited several disadvantages, such as catalyst deactivation, excessive hydrogenation, the formation of green oil or carbon, temperature overheating issues, or low yield per unit reactor volume.

Therefore, in the relevant field, there is a need for the development of improved methods and reactors that enable more efficient and stable production of aromatic compounds and C2 hydrocarbons, particularly from methane.

### CITATION LIST

### Patent Literature

(Patent Document 1) U.S. Pat. No. 4,424,401 (Registration Date: Jan. 3, 1984)
(Patent Document 2) U.S. Pat. No. 8,013,196 (Date of Registration: Sep. 6, 2011)

### Non-Patent Literature

(Non-Patent Document 1) X, Guo et al., Direct, Nonoxidative Conversion of Methane to Ethylene, Aromatics, and Hydrogen, Science, 344, 2014, 616˜619
(Non-Patent Document 2) Mann Sakbodin et al., Hydrogen-Permeable Tubular Membrane Reactor: Promoting Conversion and Product Selectivity for Nonoxidative Activation of Methane over an FeVSiO2 Catalyst, Angew. Chem. 2016, 128, 16383-16386

### DISCLOSURE

### Technical Problem

The main purpose of the present invention is to solve the aforementioned problems and significantly improve the conversion efficiency of methane in the production of C2 hydrocarbons and aromatic compounds. It also aims to provide high yields of C2 hydrocarbons and aromatic compounds. Furthermore, the invention seeks to offer a method for manufacturing a non-oxidative direct conversion reactor and a process for producing C2 hydrocarbons and aromatic compounds using the same.

### Technical Solution

To achieve the above objectives, one embodiment of the present invention provides a non-oxidative direct conversion reactor for comprising: methane, an inlet section for introducing a methane-containing feedstock, a reaction section for reacting the methane-containing feedstock introduced from the inlet section to produce a product containing C2 hydrocarbon compounds and aromatic compounds, and an outlet section for discharging the product containing C2 hydrocarbon compounds and aromatic compounds generated in the reaction section. The reaction section is divided into: a preheating section, which reacts the methane-containing feedstock introduced from the inlet section in the presence of a catalyst for dehydroaromatization to produce unsaturated hydrocarbons containing aromatic compounds and unreacted components; and a reaction zone, which reacts the unsaturated hydrocarbons containing aromatic compounds and unreacted components produced in the preheating section at 900° C. to 1,200° C. to generate hydrogen, C2 hydrocarbon compounds, and aromatic compounds.

In a preferred embodiment of the present invention, the catalyst for dehydroaromatization may comprise: zeolite; and at least one transition metal selected from the group consisting of transition metals of the 6th period in the periodic table and transition metals of Groups 4 to 6 in the periodic table, which are supported on the zeolite.

In a preferred embodiment of the present invention, the transition metal may be at least one selected from the group consisting of molybdenum, nickel, iron, and platinum.

In a preferred embodiment of the present invention, the zeolite may have an SiO₂/Al₂O₃ molar ratio of 1 to 300.

In a preferred embodiment of the present invention, the catalyst for dehydroaromatization may contain 1 wt % to 8 wt % of a transition metal based on the total weight of the catalyst.

In a preferred embodiment of the present invention, the reaction in the preheating section may be carried out at a temperature of 600° C. to 800° C.

In a preferred embodiment of the present invention, the reactor may be characterized in that the ratio of the space velocity (GHSV) in the reaction zone to the space velocity (WHSV) of the dehydroaromatization catalyst in the preheating section is 0.01 g·ml⁻¹ to 6 g·ml⁻¹.

In a preferred embodiment of the present invention, the space velocity (WHSV) in the preheating section may be 2,000 ml·h⁻¹·g_{cat}⁻¹ to 12,000 ml·h⁻¹·g_{cat}⁻¹.

In a preferred embodiment of the present invention, the C2 hydrocarbon compound may be at least one selected from the group consisting of ethane, ethylene, and acetylene.

Another embodiment of the present invention provides a method for producing C2 hydrocarbon compounds and aromatic compounds from methane using the above-described non-oxidative direct conversion reactor for methane.

### Advantageous Effects

According to the present invention, in the production of C2 hydrocarbon compounds and aromatic compounds from methane, methane consumption can be significantly improved, and high yields of C2 hydrocarbons and aromatic compounds can be achieved.

### BRIEF DESCRIPTION OF DRAWINGS

FIG. 1 shows a schematic longitudinal sectional view of a non-oxidative direct conversion reactor for methane according to one embodiment of the present invention.

FIG. 2 a schematic longitudinal sectional view of a non-oxidative direct conversion reactor for methane according to another embodiment of the present invention.

### DESCRIPTION OF SYMBOLS

100: Non-oxidative direct conversion reactor for methane
110: Inlet section
111: Catalyst for dehydroaromatization
120: Reaction section
121: Preheating section
122: Reaction zone
130: Outlet section

### MODE FOR DISCLOSURE

Unless otherwise defined, all technical and scientific terms used in this specification have the same meanings as commonly understood by those skilled in the art to which this invention pertains. In general, the nomenclature used in this specification is well known and commonly employed in the art.

Throughout this specification, when a part is described as “including” a certain component, it means that, unless specifically stated otherwise, other components may be additionally included rather than excluded.

The terms “comprises,” “includes,” or “has,” as used in this specification, indicate the presence of features, numerals, steps, operations, components, parts, or combinations thereof described in the specification, and do not preclude the possibility that other features, numerals, steps, operations, components, parts, or combinations thereof not mentioned may exist or be added.

Throughout this specification, the terms “reaction section” or “reaction zone” refer to the space within the reactor where reactants undergo a reaction. The terms “inner” and “inner side” refer to the direction toward the radial center of a circle in a cross-section of the reactor cut perpendicularly to the direction of gravity, while the terms “outer” or “outer side” refer to the direction toward the radial circumference of the circle in the same cross-section.

In one aspect, the present invention relates to a non-oxidative direct conversion reactor for methane, comprising: an inlet section for introducing a methane-containing feedstock; a reaction section for reacting the methane-containing feedstock introduced from the inlet section to produce a product containing C2 hydrocarbon compounds and aromatic compounds; and an outlet section for discharging the product containing C2 hydrocarbon compounds and aromatic compounds generated in the reaction section. The reaction section is divided into: a preheating section, which reacts the methane-containing feedstock introduced from the inlet section in the presence of a catalyst for dehydroaromatization to produce unsaturated hydrocarbons containing aromatic compounds and unreacted components; and a reaction zone, which reacts the unsaturated hydrocarbons containing aromatic compounds and unreacted components produced in the preheating section at 900° C. to 1,200° C. to generate hydrogen, C2 hydrocarbon compounds, and aromatic compounds.

More specifically, the non-oxidative direct conversion reaction of methane proceeds under high temperature and high pressure due to the low reactivity of methane. As a result, there is a rapid decline in catalyst activity caused by carbon (coke) deposition on the catalyst, which leads to problems such as low methane conversion rates and poor yields of C2 hydrocarbon compounds and aromatic compounds.

In this invention, the reaction section of the non-oxidative direct conversion reactor for methane is divided into a preheating section and a reaction zone. In the preheating section, the dehydroaromatization reaction of methane is carried out at a lower temperature than in the reaction zone to produce unsaturated hydrocarbons containing aromatic compounds. In the reaction zone, the unsaturated hydrocarbons containing aromatic compounds, which are hydrogen radical donors generated in the preheating section, are introduced and utilized as catalysts to activate the C—H bonds of methane. This approach significantly improves methane consumption and enhances the performance of the non-oxidative direct conversion reactor for methane. The linkage of these non-oxidative methane conversion reactions makes the process more spontaneous and reduces the required energy, thereby improving the efficiency of the non-oxidative direct conversion reaction of methane.

Hereinafter, the present invention will be described in detail with reference to the accompanying drawings.

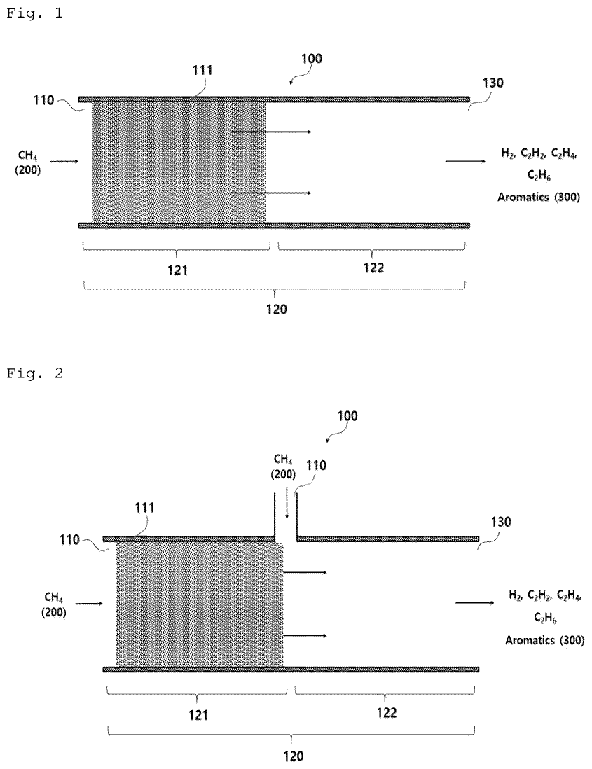

FIG. 1 is a schematic longitudinal sectional view of a non-oxidative direct conversion reactor for methane according to one embodiment of the present invention, and FIG. 2 is a schematic longitudinal sectional view of a non-oxidative direct conversion reactor for methane according to another embodiment of the present invention. Referring to FIGS. 1 and 2, the non-oxidative direct conversion reactor for methane (100) according to the present invention includes an inlet section (110) through which a methane-containing feedstock (200) is introduced, a reaction section (120) that produces a product (300) containing hydrogen, C2 hydrocarbon compounds, and aromatic compounds through the reaction of the methane-containing feedstock introduced from the inlet section (110), and an outlet section (130) that discharges the product (300) containing hydrogen, C2 hydrocarbon compounds, and aromatic compounds generated in the reaction section (120).

The reactor (100) may have variable dimensions or shapes depending on production capacity, feed rate, and catalyst, and it can be adjusted using various methods known to those skilled in the art. Preferably, it is a tubular reactor, with an inlet section (110) formed on one side to introduce the methane-containing feedstock (200). The introduced methane-containing feedstock (200) undergoes a reaction in the reaction section (120). On the opposite side of the inlet section, an outlet section (130) is formed to discharge the product (300), containing hydrogen, C2 hydrocarbon compounds, and aromatic compounds, which is generated after the reaction is completed, either to the outside or to a subsequent stage.

The inlet section (110) of the reactor can be arranged in multiple numbers and positioned in various locations, such as the upper, lower, right, or left side of the reactor, without restriction. To maximize reactivity while reducing the concentration of radical donors and coke formation in the preheating section (121), which will be described later, an additional inlet section (110) can preferably be arranged, as shown in FIG. 2, between the preheating section (121) and the reaction zone (122) to introduce the methane-containing feedstock.

Meanwhile, the outlet section (130) can also be arranged in multiple numbers on the other side of the inlet section (110) corresponding to its position.

The methane-containing feedstock (200) introduced through the inlet section (110) can be used without limitation as long as it is a mixture containing methane. For example, it may include shale gas, natural gas, and the like. Preferably, in addition to methane, it may contain inert gases and/or non-inert gases.

The methane contained in the methane-containing feedstock may be 2% (v/v) or more of the total volume of the methane-containing feedstock supplied into the reactor, and more preferably, it may range from 40% (v/v) to 100% (v/v). The inert gas and/or non-inert gas may be 98% (v/v) or less of the total volume of the methane-containing feedstock, and more preferably, it may be 60% (v/v) or less.

The inert gas and/or non-inert gas functions to stably generate and sustain the reaction state. The inert gases may include nitrogen, helium, neon, argon, or krypton, while the non-inert gases may include carbon monoxide, hydrogen, carbon dioxide, water, ethane, ethylene, propane, propylene, butane, butylene, primary alcohols (C₁-C₅), secondary alcohols (C2-C5), or alkanes (C2-C8). Preferably, the inert and non-inert gases may include argon, nitrogen, hydrogen, oxygen, and water.

The methane-containing feedstock introduced into the reaction section (120) through the inlet section (110) sequentially passes through the preheating section (121) and the reaction zone (122), producing a product (300) containing hydrogen, C2 hydrocarbon compounds, and aromatic compounds.

In the preheating section (121), the introduced methane-containing feedstock undergoes a non-oxidative dehydroaromatization reaction, as shown in Reaction Formula 1, to produce aromatic compounds and hydrogen.

6CH₄→C₆H₆+9H₂ [Reaction Formula 1]

At this time, the reaction in the preheating section (121) can be carried out at a temperature of 600° C. to 800° C., preferably 650° C. to 750° C., and at a pressure of 0.1 bar to 10 bar, preferably 0.1 bar to 5 bar.

The range of the above reaction conditions is determined considering the methane conversion rate and the yield of aromatic compounds, offering the advantage of maximizing the selectivity of methane to aromatic compounds. In other words, under these conditions, the formation of paraffins, olefins, and coke is minimized, thereby supplying the necessary concentration of hydrogen radical donors for the reaction zone while simultaneously minimizing pressure buildup caused by coke formation during the reaction and reducing carbon efficiency due to coke generation.

If the reaction temperature in the preheating section (121) is below 600° C., the catalytic activity for the reaction may be too low, resulting in a significantly reduced yield of aromatic compounds. On the other hand, if the temperature exceeds 800° C., the deactivation rate of the dehydrogenation catalyst due to coke formation increases rapidly. As a result, it becomes necessary to minimize the residence time of methane in the reactor to suppress coke formation, and there may be an issue of requiring a large amount of energy for heating the reactor.

In addition, if the reaction pressure in the preheating section (121) is below 1 bar, coke formation can be suppressed, but the energy efficiency may be low due to reduced methane activation. Conversely, if the pressure exceeds 10 bar, coke formation is promoted, leading to issues where the reactor's residence time and product cooling must be efficiently designed.

The space velocity (Weight Hourly Space Velocity: WHSV) in the preheating section is set to 2,000 mlh⁻¹g_{cat}⁻¹ to 12,000 mlh⁻¹g_{cat}⁻¹. When the space velocity in the preheating section satisfies this range, it is possible to regulate the chain reactivity during the reaction, from methane to ethane and ethylene, as well as from ethane and ethylene to aromatic compounds, thereby maximizing the production of aromatic compounds. Furthermore, it can minimize coke formation from aromatic compounds caused by excessive reactions.

At this time, the space velocity (Weight Hourly Space Velocity: WHSV) in the preheating section refers to the flow rate of reaction gas within the preheating section divided by the reactor volume and can be calculated as the gas volumetric flow rate (ml/hr) divided by the catalyst weight (g).

Meanwhile, the preheating section may include a dehydroaromatization reaction catalyst (111) capable of promoting the dehydroaromatization reaction of methane. The dehydroaromatization reaction catalyst may consist of a catalytic active component supported on a catalyst support that provides acid sites capable of inducing the dehydroaromatization reaction.

The support providing the acid sites may be a metal silicate, preferably a zeolite, and more preferably a zeolite with an MFI structure such as Silicalite-1, ZSM-5, or TS-1. In this case, the zeolite may include not only Al but also additional elements from a third main group, such as Ga, B, or In. Furthermore, non-limiting examples of dopants may include noble metals such as Rh, Pd, Ag, Ir, Pt, and/or Au, or transition metals such as Fe, Ni, Co, and/or Cu.

In addition, the SiO₂/Al₂O₃ molar ratio of the zeolite ranges from 1 to 300, preferably from 10 to 100, and more preferably from 10 to 70. When a zeolite within this range is used, it is possible to relatively increase the concentration of Brønsted acid sites compared to Lewis acid sites. This provides acid sites that can selectively convert ethane and ethylene compounds into aromatic compounds while simultaneously enhancing the dispersion of the catalytic active components on the surface of the zeolite.

Meanwhile, non-limiting examples of the catalytic active components may include one or more transition metals selected from the group consisting of 6th-period transition metals in the periodic table, such as molybdenum, nickel, iron, and platinum, and group 4 to 6 transition metals in the periodic table. Preferably, it may include molybdenum (Mo). The molybdenum can be supported with high dispersion during catalyst preparation while neutralizing the strong Brønsted acid sites of the catalyst support. During the reaction, it is activated into molybdenum carbide, which enables the initial activation of methane and its subsequent conversion into ethane and ethylene. Therefore, for the catalyst to exhibit reactivity, a sufficient number of molybdenum active sites capable of interacting with the acid sites of the catalyst support must be present.

The supported amount of the catalytic active component, i.e., the transition metal, may range from 1 wt % to 8 wt % relative to the total weight of the catalyst, and preferably from 4 wt % to 7 wt %. The dehydroaromatization reaction catalyst within this range can maximize surface active sites for the initial activation of methane while suppressing the formation of coke containing polyaromatic compounds at the acid sites, thereby increasing the selectivity for aromatic compounds.

As described above, the methane-containing feedstock undergoes a reaction in the preheating section (121), producing aromatic compounds, unsaturated hydrocarbons containing aromatic compounds that act as hydrogen radical donors, and unreacted components. These are then introduced into the reaction zone (122). The unreacted components, i.e., methane, introduced into the reaction zone (122) is activated by the aromatic compounds that are simultaneously introduced into the reaction zone (122), as shown in Reaction Formula 2, and is synthesized into C2 hydrocarbon compounds (ethylene, acetylene, and ethane) and aromatic compounds. At this time, the aromatic compounds reversibly provide and accept hydrogen radicals, thereby promoting the initial activation of methane.

CH₄+C₆H₆→CH_{3′}+C₆H_{5′}+H₂

2CH_{3′}→C₂H₆

C₂H₆→C₂H₄+H₂

C₂H₄→C₂H₂+2H₂

3C₂H₂→C₆H₆

2C₆H_{5′}++H₂→2C₂H₆ [Reaction Formula 2]

The reaction of the methane mixture containing aromatic compounds, which act as hydrogen radical donors in the reaction zone (122), can be carried out at a temperature of 900° C. to 1,200° C., preferably 950° C. to 1, 150° C., and at a pressure of 0.1 bar to 10 bar, preferably 0.1 bar to 5 bar. The space velocity in the reaction zone may range from 200 h⁻¹ to 12,000 h⁻¹.

The above reaction conditions are determined with consideration for the selectivity and yield of hydrocarbons, offering the advantage of maximizing the selectivity of methane to hydrocarbons. In other words, under these conditions, coke formation is minimized, thereby reducing pressure drop caused by coke formation during the reaction and minimizing carbon deposition resulting from coke generation.

If the reaction temperature in the reaction zone (122) is below 900° C., the reactivity may be low, leading to the issue of requiring a larger reactor size for the reaction zone compared to the preheating section. On the other hand, if the temperature exceeds 1,200° C., side reactions (dehydrogenation and coupling) may become dominant, thereby promoting coke formation.

In addition, if the reaction pressure in the reaction zone (122) is below 0.1 bar, the reactivity may be low, resulting in the issue of requiring a larger reactor size for the reaction zone compared to the preheating section. Conversely, if the pressure exceeds 10 bar, side reactions (dehydrogenation and coupling) may dominate, thereby promoting coke formation.

The reaction zone may have a ratio of the space velocity (GHSV) in the reaction zone to the space velocity (WHSV) of the preheating section catalyst ranging from 0.01 gml⁻¹ to 6 gml⁻¹. If the ratio of the space velocity (GHSV) in the reaction zone to the space velocity (WHSV) of the preheating section catalyst is below 0.01 gml⁻¹, side reactions (dehydrogenation and coupling) may dominate, thereby promoting coke formation. On the other hand, if the ratio exceeds 6 gml⁻¹, the reaction temperature in the reaction zone must be increased compared to that of the preheating section, leading to a decrease in thermal efficiency.

The product containing the C2 hydrocarbon compounds, aromatic compounds synthesized in this manner, and hydrogen is discharged to the exterior or downstream through the outlet section (130) of the reactor. At this time, the C2 hydrocarbon compounds may include ethane, ethylene, and acetylene, while the aromatic compounds may include benzene, toluene, xylene, ethylbenzene, and naphthalene aromatic compounds.

The non-oxidative direct conversion reactor for methane according to the present invention utilizes the unsaturated hydrocarbons containing aromatic compounds, produced by the dehydroaromatization reaction in the preheating section, in the non-oxidative methane conversion reaction in the reaction zone. This approach reduces the energy and reactor volume required for methane conversion in the reaction zone due to the dehydroaromatization reaction in the preheating section and has the effect of lowering the reaction temperature.

From another perspective, the present invention relates to a method for producing C2 hydrocarbon compounds and aromatic compounds from methane using the non-oxidative direct conversion reactor for methane.

The method for producing C2 hydrocarbon compounds and aromatic compounds according to the present invention involves supplying a methane-containing feedstock (200) through the inlet section (110) of the non-oxidative direct conversion reactor (100) for methane described above. In the preheating section (121) of the reactor, a dehydroaromatization reaction generates unsaturated hydrocarbons containing aromatic compounds, which act as hydrogen radical donors, along with unreacted components. The generated unsaturated hydrocarbons containing aromatic compounds and the unreacted components are introduced into the reaction zone (122). In the reaction zone (122), the unreacted components introduced are selectively activated at their C—H bonds by hydrogen radicals derived from the aromatic compounds introduced simultaneously, promoting the formation of methyl radicals. These methyl radicals undergo a series of chain reactions in the gas phase to produce C2 hydrocarbon compounds and aromatic compounds, which are then discharged through the outlet section (130).

The method for producing C2 hydrocarbon compounds and aromatic compounds according to the present invention is as described in the corresponding non-oxidative direct conversion reactor for methane. Therefore, a person skilled in the art will be able to clearly understand the method, and further explanation is omitted here to avoid redundancy.

Hereinafter, the present invention will be described in more detail through specific examples. The following examples are merely provided to aid in understanding the present invention and are not intended to limit the scope of the invention.

### Preparation Example 1: Preparation of a Catalyst for Dehydroaromatization Reaction

The 6 wt % Mo/H-ZSM-5 (SiO₂/Al₂O₃=30) catalyst was prepared using the wet impregnation method. First, 1.15 g of ammonium heptamolybdate [(NH₄)₆Mo₇O₂₄·4H₂O] as a molybdenum precursor was dissolved in 20 g of deionized water (D.I. water). Then, 10 g of H-ZSM-5 (SiO₂/Al₂O₃=30) was added to the solution and maintained at 40° C. for 30 minutes. Subsequently, the water was removed at 50° C. using a rotary vacuum evaporator (RV 10 control, IKA) to obtain a solid. The obtained solid was dried at 110° C. for 10 hours and then calcined in an air atmosphere at 500° C. to prepare the catalyst.

### Preparation Example 2: Preparation of a Catalyst for Dehydroaromatization Reaction

The 6 wt % Mo/H-ZSM-5 (SiO₂/Al₂O₃=50) catalyst was prepared using the wet impregnation method. First, 1.15 g of ammonium heptamolybdate [(NH₄)₆MO₇O₂₄·4H₂O] as a molybdenum precursor was dissolved in 20 g of deionized water (D.I. water). Then, 10 g of H-ZSM-5 (SiO₂/Al₂O₃=50) was added to the solution and maintained at 40° C. for 30 minutes. Subsequently, the water was removed at 50° C. using a rotary vacuum evaporator (RV 10 control, IKA) to obtain a solid. The obtained solid was dried at 110° C. for 10 hours and then calcined in an air atmosphere at 500° C. to prepare the catalyst.

### Preparation Example 3: Preparation of a Catalyst for Dehydroaromatization Reaction

The 6 wt % Mo/H-ZSM-5 (SiO₂/Al₂O₃=80) catalyst was prepared using the wet impregnation method. First, 1.15 g of ammonium heptamolybdate [(NH₄)₆MO₇O₂₄·4H₂O] as a molybdenum precursor was dissolved in 20 g of deionized water (D.I. water). Then, 10 g of H-ZSM-5 (SiO₂/Al₂O₃=80) was added to the solution and maintained at 40° C. for 30 minutes. Subsequently, the water was removed at 50° C. using a rotary vacuum evaporator (RV 10 control, IKA) to obtain a solid. The obtained solid was dried at 110° C. for 10 hours and then calcined in an air atmosphere at 500° C. to prepare the catalyst.

### Preparation Example 4: Preparation of a Catalyst for Dehydroaromatization Reaction

The 6 wt % Mo/H-ZSM-5 (SiO₂/Al₂O₃=280) catalyst was prepared using the wet impregnation method. First, 1.15 g of ammonium heptamolybdate [(NH₄)₆Mo₇O₂₄·4H₂O] as a molybdenum precursor was dissolved in 20 g of deionized water (D.I. water). Then, 10 g of H-ZSM-5 (SiO₂/Al₂O₃=280) was added to the solution and maintained at 40° C. for 30 minutes. Subsequently, the water was removed at 50° C. using a rotary vacuum evaporator (RV 10 control, IKA) to obtain a solid. The obtained solid was dried at 110° C. for 10 hours and then calcined in an air atmosphere at 500° C. to prepare the catalyst.

### <Preparation Example 5: Preparation of a Catalyst for Dehydroaromatization Reaction

The 1 wt % Mo/H-ZSM-5 (SiO₂/Al₂O₃=30) catalyst was prepared using the wet impregnation method. First, 0.22 g of ammonium heptamolybdate [(NH₄)₆MO₇O₂₄·4H₂O] as a molybdenum precursor was dissolved in 20 g of deionized water (D.I. water). Then, 10 g of H-ZSM-5 (SiO₂/Al₂O₃=30) was added to the solution and maintained at 40° C. for 30 minutes. Subsequently, the water was removed at 50° C. using a rotary vacuum evaporator (RV 10 control, IKA) to obtain a solid. The obtained solid was dried at 110° C. for 10 hours and then calcined in an air atmosphere at 500° C. to prepare the catalyst.

### Preparation Example 6: Preparation of a Catalyst for Dehydroaromatization Reaction

The 3 wt % Mo/H-ZSM-5 (SiO₂/Al₂O₃=30) catalyst was prepared using the wet impregnation method. First, 0.57 g of ammonium heptamolybdate [(NH₄) (MO₇O₂₄·4H₂O] as a molybdenum precursor was dissolved in 20 g of deionized water (D.I. water). Then, 10 g of H-ZSM-5 (SiO₂/Al-03=30) was added to the solution and maintained at 40° C. for 30 minutes. Subsequently, the water was removed at 50° C. using a rotary vacuum evaporator (RV 10 control, IKA) to obtain a solid. The obtained solid was dried at 110° C. for 10 hours and then calcined in an air atmosphere at 500° C. to prepare the catalyst.

### Preparation Example 7: Preparation of a Catalyst for Dehydroaromatization Reaction

The 8 wt % Mo/H-ZSM-5 (SiO₂/Al₂O₃=30) catalyst was prepared using the wet impregnation method. First, 1.60 g of ammonium heptamolybdate [(NH₄)₆Mo₇O₂₄·4H₂O] as a molybdenum precursor was dissolved in 20 g of deionized water (D.I. water). Then, 10 g of H-ZSM-5 (SiO₂/Al₂O₃=30) was added to the solution and maintained at 40° C. for 30 minutes. Subsequently, the water was removed at 50° C. using a rotary vacuum evaporator (RV 10 control, IKA) to obtain a solid. The obtained solid was dried at 110° C. for 10 hours and then calcined in an air atmosphere at 500° C. to prepare the catalyst.

### Preparation Example 8: Preparation of a Catalyst for Dehydroaromatization Reaction

The 1 wt % Mo/H-ZSM-5 (SiO₂/Al₂O₃=50) catalyst was prepared using the wet impregnation method. First, 0.22 g of ammonium heptamolybdate [(NH₄)₆MO₇O₂₄·4H₂O] as a molybdenum precursor was dissolved in 20 g of deionized water (D.I. water). Then, 10 g of H-ZSM-5 (SiO₂/Al₂O₃=50) was added to the solution and maintained at 40° C. for 30 minutes. Subsequently, the water was removed at 50° C. using a rotary vacuum evaporator (RV 10 control, IKA) to obtain a solid. The obtained solid was dried at 110° C. for 10 hours and then calcined in an air atmosphere at 500° C. to prepare the catalyst.

### Preparation Example 9: Preparation of a Catalyst for Dehydroaromatization Reaction

The 3 wt % Mo/H-ZSM-5 (SiO₂/Al₂O₃=50) catalyst was prepared using the wet impregnation method. First, 0.57 g of ammonium heptamolybdate [(NH₄)₆Mo₇O₂₄·4H₂O] as a molybdenum precursor was dissolved in 20 g of deionized water (D.I. water). Then, 10 g of H-ZSM-5 (SiO₂/Al₂O₃=50) was added to the solution and maintained at 40° C. for 30 minutes. Subsequently, the water was removed at 50° C. using a rotary vacuum evaporator (RV 10 control, IKA) to obtain a solid. The obtained solid was dried at 110° C. for 10 hours and then calcined in an air atmosphere at 500° C. to prepare the catalyst.

### Preparation Example 10: Preparation of a Catalyst for Dehydroaromatization Reaction

The 8 wt % Mo/H-ZSM-5 (SiO₂/Al₂O₃=50) catalyst was prepared using the wet impregnation method. First, 1.60 g of ammonium heptamolybdate [(NH₄)₆MO₇O₂₄·4H₂O] as a molybdenum precursor was dissolved in 20 g of deionized water (D.I. water). Then, 10 g of H-ZSM-5 (SiO₂/Al₂O₃=50) was added to the solution and maintained at 40° C. for 30 minutes. Subsequently, the water was removed at 50° C. using a rotary vacuum evaporator (RV 10 control, IKA) to obtain a solid. The obtained solid was dried at 110° C. for 10 hours and then calcined in an air atmosphere at 500° C. to prepare the catalyst.

### Preparation Example 11: Preparation of a Catalyst for Dehydroaromatization Reaction

The 1 wt % Mo/H-ZSM-5 (SiO₂/Al₂O₃=80) catalyst was prepared using the wet impregnation method. First, 0.22 g of ammonium heptamolybdate [(NH₄)₆Mo₇O₂₄·4H₂O] as a molybdenum precursor was dissolved in 20 g of deionized water (D.I. water). Then, 10 g of H-ZSM-5 (SiO₂/Al₂O₃=80) was added to the solution and maintained at 40° C. for 30 minutes. Subsequently, the water was removed at 50° C. using a rotary vacuum evaporator (RV 10 control, IKA) to obtain a solid. The obtained solid was dried at 110° C. for 10 hours and then calcined in an air atmosphere at 500° C. to prepare the catalyst.

### Preparation Example 12: Preparation of a Catalyst for Dehydroaromatization Reaction

The 3 wt % Mo/H-ZSM-5 (SiO₂/Al₂O₃=80) catalyst was prepared using the wet impregnation method. First, 0.57 g of ammonium heptamolybdate [(NH₄)₆MO₇O₂₄·4H₂O] as a molybdenum precursor was dissolved in 20 g of deionized water (D.I. water). Then, 10 g of H-ZSM-5 (SiO₂/Al₂O₃=80) was added to the solution and maintained at 40° C. for 30 minutes. Subsequently, the water was removed at 50° C. using a rotary vacuum evaporator (RV 10 control, IKA) to obtain a solid. The obtained solid was dried at 110° C. for 10 hours and then calcined in an air atmosphere at 500° C. to prepare the catalyst.

### Preparation Example 13: Preparation of a Catalyst for Dehydroaromatization Reaction

The 8 wt % Mo/H-ZSM-5 (SiO₂/Al₂O₃=80) catalyst was prepared using the wet impregnation method. First, 1.60 g of ammonium heptamolybdate [(NH₄)₆Mo₇O₂₄·4H₂O] as a molybdenum precursor was dissolved in 20 g of deionized water (D.I. water). Then, 10 g of H-ZSM-5 (SiO₂/Al₂O₃=80) was added to the solution and maintained at 40° C. for 30 minutes. Subsequently, the water was removed at 50° C. using a rotary vacuum evaporator (RV 10 control, IKA) to obtain a solid. The obtained solid was dried at 110° C. for 10 hours and then calcined in an air atmosphere at 500° C. to prepare the catalyst.

### Examples 1 to 13

Using the non-oxidative direct conversion reactor for methane, as shown in FIG. 1, C2 hydrocarbon compounds and aromatic compounds were produced from methane. The non-oxidative direct conversion reactor for methane was a fixed-bed reactor, with a quartz tubular reactor of 4 mm inner diameter installed in a furnace having two separate heating zones, each 10 cm in height. The upper section was designated as the preheating section, and the lower section as the reaction zone. Catalysts prepared in the preparation examples were crushed to a particle size of 425 μm to 850 μm, and 0.20 g was packed into the preheating section. A methane-containing feedstock (90% (v/v) CH₄, 10% (v/v) Ar) was introduced into the reactor at a flow rate of 20 ml/min using mass flow controllers, and the reaction was carried out under the conditions listed in Table 1. Argon (Ar) gas was used as an internal standard.

The resulting products were analyzed every hour starting 30 minutes after the introduction of the reactants using an Agilent 8890 GC connected online. The gaseous products were analyzed using a Thermal Conductivity Detector (TCD) connected to a ShinCarbon ST 80/100 column and two Flame Ionization Detectors (FID), each connected to an Rt-alumina BOND column and an RTx-VMS column, respectively. Hydrogen, methane, argon, and C2 hydrocarbon compounds (ethane, ethylene, and acetylene) were separated on the ShinCarbon ST 80/100 column and detected with the TCD. The conversion rate was calculated based on the ratio of methane peak area to the peak area of argon, which was used as an internal standard. Light hydrocarbons in the C1 to C5 range and benzene were separated on the Rt-alumina BOND column and detected with the FID, while aromatic compounds, including benzene, were separated on the RTx-VMS column and detected with the FID. All gases were quantified using standard samples. The amounts of methane consumed (C-mmol) and products generated (C-mmol) were obtained by integrating the detected quantities over the reaction time from 1.5 to 10.5 h, and the results are shown in Table 2.

### Comparative Example 1

C2 hydrocarbon compounds and aromatic compounds were produced in the same manner as in Example 1, except that no catalyst was used in the preheating section and the reaction was carried out under the conditions specified in Table 1, with the temperature of the preheating section set to 700° C. and the temperature of the reaction zone set to 200° C. The resulting products were then analyzed using the same method as in Example 1, and the results are shown in Table 2.

### Comparative Example 2

C2 hydrocarbon compounds and aromatic compounds were produced in the same manner as in Example 1, except that the reaction was carried out under the conditions specified in Table 1, with the temperature of the preheating section set to 700° C. and the temperature of the reaction zone set to 200° C. The resulting products were then analyzed using the same method as in Example 1, and the results are presented in Table 2.

### Comparative Example 3

C2 hydrocarbon compounds and aromatic compounds were produced in the same manner as in Example 2, except that the reaction was carried out under the conditions specified in Table 1, with the temperature of the preheating section set to 700° C. and the temperature of the reaction zone set to 200° C. The resulting products were then analyzed using the same method as in Example 1, and the results are presented in Table 2.

### Comparative Example 4

C2 hydrocarbon compounds and aromatic compounds were produced in the same manner as in Example 3, except that the reaction was carried out under the conditions specified in Table 1, with the temperature of the preheating section set to 700° C. and the temperature of the reaction zone set to 200° C. The resulting products were then analyzed using the same method as in Example 1, and the results are presented in Table 2.

### Comparative Example 5

C2 hydrocarbon compounds and aromatic compounds were produced in the same manner as in Example 4, except that the reaction was carried out under the conditions specified in Table 1, with the temperature of the preheating section set to 700° C. and the temperature of the reaction zone set to 200° C. The resulting products were then analyzed using the same method as in Example 1, and the results are presented in Table 2.

### Comparative Example 6

C2 hydrocarbon compounds and aromatic compounds were produced in the same manner as in Example 5, except that the reaction was carried out under the conditions specified in Table 1, with the temperature of the preheating section set to 700° C. and the temperature of the reaction zone set to 200° C. The resulting products were then analyzed using the same method as in Example 1, and the results are presented in Table 2.

### Comparative Example 7

C2 hydrocarbon compounds and aromatic compounds were produced in the same manner as in Example 6, except that the reaction was carried out under the conditions specified in Table 1, with the temperature of the preheating section set to 700° C. and the temperature of the reaction zone set to 200° C. The resulting products were then analyzed using the same method as in Example 1, and the results are presented in Table 2.

### Comparative Example 8

C2 hydrocarbon compounds and aromatic compounds were produced in the same manner as in Example 7, except that the reaction was carried out under the conditions specified in Table 1, with the temperature of the preheating section set to 700° C. and the temperature of the reaction zone set to 200° C. The resulting products were then analyzed using the same method as in Example 1, and the results are presented in Table 2.

### Comparative Example 9

C2 hydrocarbon compounds and aromatic compounds were produced in the same manner as in Example 8, except that the reaction was carried out under the conditions specified in Table 1, with the temperature of the preheating section set to 700° C. and the temperature of the reaction zone set to 200° C. The resulting products were then analyzed using the same method as in Example 1, and the results are presented in Table 2.

### Comparative Example 10

C2 hydrocarbon compounds and aromatic compounds were produced in the same manner as in Example 9, except that the reaction was carried out under the conditions specified in Table 1, with the temperature of the preheating section set to 700° C. and the temperature of the reaction zone set to 200° C. The resulting products were then analyzed using the same method as in Example 1, and the results are presented in Table 2.

### Comparative Example 11

C2 hydrocarbon compounds and aromatic compounds were produced in the same manner as in Example 10, except that the reaction was carried out under the conditions specified in Table 1, with the temperature of the preheating section set to 700° C. and the temperature of the reaction zone set to 200° C. The resulting products were then analyzed using the same method as in Example 1, and the results are presented in Table 2.

### Comparative Example 12

C2 hydrocarbon compounds and aromatic compounds were produced in the same manner as in Example 11, except that the reaction was carried out under the conditions specified in Table 1, with the temperature of the preheating section set to 700° C. and the temperature of the reaction zone set to 200° C. The resulting products were then analyzed using the same method as in Example 1, and the results are presented in Table 2.

### Comparative Example 13

C2 hydrocarbon compounds and aromatic compounds were produced in the same manner as in Example 12, except that the reaction was carried out under the conditions specified in Table 1, with the temperature of the preheating section set to 700° C. and the temperature of the reaction zone set to 200° C. The resulting products were then analyzed using the same method as in Example 1, and the results are presented in Table 2.

### Comparative Example 14

C2 hydrocarbon compounds and aromatic compounds were produced in the same manner as in Example 13, except that the reaction was carried out under the conditions specified in Table 1, with the temperature of the preheating section set to 700° C. and the temperature of the reaction zone set to 200° C. The resulting products were then analyzed using the same method as in Example 1, and the results are presented in Table 2.

### Comparative Example 15

C2 hydrocarbon compounds and aromatic compounds were produced in the same manner as in Example 1, except that the reaction was carried out without using a catalyst in the preheating section under the conditions specified in Table 1. The resulting products were then analyzed using the same method as in Example 1, and the results are presented in Table 2.

### Comparative Example 16

C2 hydrocarbon compounds and aromatic compounds were produced in the same manner as in Example 1, except that the reaction was carried out without using a catalyst in the preheating section and with the temperature of the preheating section set to 800° C. under the conditions specified in Table 1. The resulting products were then analyzed using the same method as in Example 1, and the results are presented in Table 2.

### Comparative Example 17

C2 hydrocarbon compounds and aromatic compounds were produced in the same manner as in Example 1, except that the reaction was carried out without using a catalyst in the preheating section and with the temperature of the preheating section set to 900° C. under the conditions specified in Table 1. The resulting products were then analyzed using the same method as in Example 1, and the results are presented in Table 2.

### Comparative Example 18

C2 hydrocarbon compounds and aromatic compounds were produced in the same manner as in Example 1, except that the reaction was carried out without using a catalyst in the 5 preheating section and with the temperature of the preheating section set to 970° C. under the conditions specified in Table 1. The resulting products were then analyzed using the same method as in Example 1, and the results are presented in Table 2.

TABLE 1

| | | | | | space |
|---|---|---|---|---|---|
| | | | | | velosity |
| | | Reaction | | WHSV | ratio |
| | | temperature | | (mlh⁻¹ | [reaction |
| | Catalyst | (° C.) | | g_{cat}⁻¹) | zone/ |
| | pre- | pre- | re- | pre- | preheating |
| | heating | heating | action | heating | section] |
| Classification | section | section | zone | section | (g_{cat} ml_{reactor}⁻¹) |
| Example 1 | Pre- | 700 | 1,020 | 6,240 | 0.16 |
| | paration | | | | |
| | Example | | | | |
| | 1 | | | | |
| Example 2 | Pre- | 700 | 1,020 | 6,240 | 0.16 |
| | paration | | | | |
| | Example | | | | |
| | 2 | | | | |
| Example 3 | Pre- | 700 | 1,020 | 6,240 | 0.16 |
| | paration | | | | |
| | Example | | | | |
| | 3 | | | | |
| Example 4 | Pre- | 700 | 1,020 | 6,240 | 0.16 |
| | paration | | | | |
| | Example | | | | |
| | 4 | | | | |
| Example 5 | Pre- | 700 | 1,020 | 6,240 | 0.16 |
| | paration | | | | |
| | Example | | | | |
| | 5 | | | | |
| Example 6 | Pre- | 700 | 1,020 | 6,240 | 0.16 |
| | paration | | | | |
| | Example | | | | |
| | 6 | | | | |
| Example 7 | Pre- | 700 | 1,020 | 6,240 | 0.16 |
| | paration | | | | |
| | Example | | | | |
| | 7 | | | | |
| Example 8 | Pre- | 700 | 1,020 | 6,240 | 0.16 |
| | paration | | | | |
| | Example | | | | |
| | 8 | | | | |
| Example 9 | Pre- | 700 | 1,020 | 6,240 | 0.16 |
| | paration | | | | |
| | Example | | | | |
| | 9 | | | | |
| Example 10 | Pre- | 700 | 1,020 | 6,240 | 0.16 |
| | paration | | | | |
| | Example | | | | |
| | 10 | | | | |
| Example 11 | Pre- | 700 | 1,020 | 6,240 | 0.16 |
| | paration | | | | |
| | Example | | | | |
| | 11 | | | | |
| Example 12 | Pre- | 700 | 1,020 | 6,240 | 0.16 |
| | paration | | | | |
| | Example | | | | |
| | 12 | | | | |
| Example 13 | Pre- | 700 | 1,020 | 6,240 | 0.16 |
| | paration | | | | |
| | Example | | | | |
| | 13 | | | | |
| Comparative | — | 700 | 200 | — | 0 |
| Example 1 | | | | | |
| Comparative | Pre- | 700 | 200 | 6,240 | 0.16 |
| Example 2 | paration | | | | |
| | Example | | | | |
| | 1 | | | | |
| Comparative | Pre- | 700 | 200 | 6,240 | 0.16 |
| Example 3 | paration | | | | |
| | Example | | | | |
| | 2 | | | | |
| Comparative | Pre- | 700 | 200 | 6,240 | 0.16 |
| Example 4 | paration | | | | |
| | Example | | | | |
| | 3 | | | | |
| Comparative | Pre- | 700 | 200 | 6,240 | 0.16 |
| Example 5 | paration | | | | |
| | Example | | | | |
| | 4 | | | | |
| Comparative | Pre- | 700 | 200 | 6,240 | 0.16 |
| Example 6 | paration | | | | |
| | Example | | | | |
| | 5 | | | | |
| Comparative | Pre- | 700 | 200 | 6,240 | 0.16 |
| Example 7 | paration | | | | |
| | Example | | | | |
| | 6 | | | | |
| Comparative | Pre- | 700 | 200 | 6,240 | 0.16 |
| Example 8 | paration | | | | |
| | Example | | | | |
| | 7 | | | | |
| Comparative | Pre- | 700 | 200 | 6,240 | 0.16 |
| Example 9 | paration | | | | |
| | Example | | | | |
| | 8 | | | | |
| Comparative | Pre- | 700 | 200 | 6,240 | 0.16 |
| Example 10 | paration | | | | |
| | Example | | | | |
| | 9 | | | | |
| Comparative | Pre- | 700 | 200 | 6,240 | 0.16 |
| Example 11 | paration | | | | |
| | Example | | | | |
| | 10 | | | | |
| Comparative | Pre- | 700 | 200 | 6,240 | 0.16 |
| Example 12 | paration | | | | |
| | Example | | | | |
| | 11 | | | | |
| Comparative | Pre- | 700 | 200 | 6,240 | 0.16 |
| Example 13 | paration | | | | |
| | Example | | | | |
| | 12 | | | | |
| Comparative | Pre- | 700 | 200 | 6,240 | 0.16 |
| Example 14 | paration | | | | |
| | Example | | | | |
| | 13 | | | | |
| Comparative | — | 700 | 1,020 | — | 0 |
| Example 15 | | | | | |
| Comparative | — | 800 | 1,020 | — | 0 |
| Example 16 | | | | | |
| Comparative | — | 900 | 1,020 | — | 0 |
| Example 17 | | | | | |
| Comparative | — | 970 | 1,020 | — | 0 |
| Example 18 | | | | | |

TABLE 2

| | Methane | Production | |
|---|---|---|---|
| | consumption | amount (C-mmol) | |
| | amount | C2 | |
| Classification | (C-mmol) | hydrocarbons | aromatics |
| Example 1 | 27.09 | 9.7 | 10.6 |
| Example 2 | 17.52 | 7.7 | 7.1 |
| Example 3 | 13.43 | 6.4 | 3.4 |
| Example 4 | 10.60 | 5.8 | 2.1 |
| Example 5 | 13.29 | 6.2 | 3.4 |
| Example 6 | 24.62 | 9.2 | 11.6 |
| Example 7 | 28.91 | 10.2 | 16.2 |
| Example 8 | 11.00 | 5.8 | 3.0 |
| Example 9 | 14.19 | 6.5 | 5.0 |
| Example 10 | 17.66 | 7.8 | 7.7 |
| Example 11 | 12.89 | 5.1 | 1.7 |
| Example 12 | 10.55 | 5.2 | 1.8 |
| Example 13 | 7.82 | 4.3 | 1.6 |
| Comparative | 1.60 | — | — |
| Example 1 | | | |
| Comparative | 12.06 | 2.3 | 7.9 |
| Example 2 | | | |
| Comparative | 6.44 | 1.6 | 4.0 |
| Example 3 | | | |
| Comparative | 4.43 | 1.2 | 2.9 |
| Example 4 | | | |
| Comparative | 3.45 | 1.8 | 0.6 |
| Example 5 | | | |
| Comparative | 1.82 | 0.9 | 1.1 |
| Example 6 | | | |
| Comparative | 8.03 | 1.8 | 6.2 |
| Example 7 | | | |
| Comparative | 14.75 | 2.5 | 12.1 |
| Example 8 | | | |
| Comparative | 2.39 | 1.0 | 1.3 |
| Example 9 | | | |
| Comparative | 4.24 | 1.2 | 3.0 |
| Example 10 | | | |
| Comparative | 5.73 | 1.5 | 4.2 |
| Example 11 | | | |
| Comparative | 3.22 | 0.8 | 0.9 |
| Example 12 | | | |
| Comparative | 5.17 | 0.8 | 1.5 |
| Example 13 | | | |
| Comparative | 2.20 | 0.9 | 1.2 |
| Example 14 | | | |
| Comparative | 4.81 | 3.3 | 0.4 |
| Example 15 | | | |
| Comparative | 4.53 | 2.8 | 0.4 |
| Example 16 | | | |
| Comparative | 6.46 | 3.9 | 0.8 |
| Example 17 | | | |
| Comparative | 12.37 | 6.8 | 2.4 |
| Example 18 | | | |

As shown in Table 2, in the cases of Examples 1 to 4, the methane consumption and the production of C2 hydrocarbon compounds and aromatic compounds were significantly higher compared to Comparative Example 1, where the reaction zone temperature was lower, and no catalyst was used in the preheating section.

On the other hand, in the cases of Comparative Examples 2 to 14, where the reaction zone temperature was lower compared to Examples 1 to 13, the methane consumption was found to be 2 to 3 times lower, and the production of C2 hydrocarbon compounds (ethane, ethylene, and acetylene) and aromatic compounds decreased under the same catalyst conditions as Examples 1 to 13. In addition, in the cases of Comparative Examples 15 to 18, where the temperature of the preheating section was varied between 700° C. and 970° C. without using a catalyst for the dehydroaromatization reaction, the methane consumption was lower compared to Examples 1 to 13, and both the methane consumption and the production of C2 hydrocarbon compounds and aromatic compounds were found to decrease. On the other hand, in the case of Comparative Example 15, where no catalyst was used in the preheating section under the same conditions as Example 1, the methane consumption did not show a significant increase.

In addition, the methane consumption and the sum of production values (C2 hydrocarbons and aromatics) for Comparative Examples 2 to 14 and Comparative Example 16, corresponding to the parallel connection of Examples 1 to 13, was found to be lower than that of Examples 1 to 13.

Through this, it was confirmed that the non-oxidative direct conversion reactor for methane according to the present invention can form an H-radical donor capable of promoting the C—H activation of methane through the dehydroaromatization reaction in the preheating section. When this is linked to the reaction in the reaction zone, methane conversion is improved, and the production of C2 hydrocarbon compounds and aromatic compounds can be increased. Additionally, it was confirmed that this approach can reduce the energy required for each heating section of the reaction and the reactor volume.

The present invention has been described with reference to the above examples and the accompanying drawings, but different embodiments may also be constructed within the concept and scope of the invention. Therefore, the scope of the present invention is defined by the appended claims and their equivalents, and is not limited to the specific embodiments described in this specification.

1. A non-oxidative direct conversion reactor for methane, comprising:
an inlet section for introducing a methane-containing feedstock;
a reaction section for reacting the methane-containing feedstock introduced from the inlet section to produce a product containing C2 hydrocarbon compounds and aromatic compounds; and
an outlet section for discharging the product containing C2 hydrocarbon compounds and aromatic compounds generated in the reaction section,
wherein the reaction section is divided into:
a preheating section that reacts the methane-containing feedstock introduced from the inlet section in the presence of a catalyst for dehydroaromatization reaction to produce unsaturated hydrocarbons containing aromatic compounds and unreacted components; and
a reaction zone that reacts the unsaturated hydrocarbons containing aromatic compounds and unreacted components generated in the preheating section at 900° C. to 1,200° C. to produce hydrogen, C2 hydrocarbon compounds, and aromatic compounds.

2. The non-oxidative direct conversion reactor for methane according to claim 1,
wherein the catalyst for the dehydroaromatization reaction comprises:
a zeolite; and one or more transition metals selected from the group consisting of 6th-period transition metals and Group 4 to 6 transition metals of the periodic table, supported on the zeolite.

3. The non-oxidative direct conversion reactor for methane according to claim 2,
wherein the transition metal is one or more selected from the group consisting of molybdenum, nickel, iron, and platinum.

4. The non-oxidative direct conversion reactor for methane according to claim 2,
wherein the zeolite has an SiO₂/Al₂O₃ molar ratio of 1 to 300.

5. The non-oxidative direct conversion reactor for methane according to claim 2,
wherein the catalyst for the dehydroaromatization reaction comprises 1 wt % to 8 wt % of the transition metal based on the total weight of the catalyst.

6. The non-oxidative direct conversion reactor for methane according to claim 1,
wherein the reaction in the preheating section is carried out at a temperature of 600° C. to 800° C.

7. The non-oxidative direct conversion reactor for methane according to claim 1,
wherein the ratio of the space velocity (GHSV) in the reaction zone to the space velocity (WHSV) of the catalyst for dehydroaromatization in the preheating section is 0.01 gml⁻¹ to 6 gml⁻¹.

8. The non-oxidative direct conversion reactor for methane according to claim 1,
wherein the space velocity (WHSV) in the preheating section is 2,000 mlh⁻¹g_{cat}⁻¹ to 12,000 mlh⁻¹g_{cat}⁻¹.

9. The non-oxidative direct conversion reactor for methane according to claim 1,
wherein the C2 hydrocarbon compounds are one or more selected from the group consisting of ethane, ethylene, and acetylene.

10. A method for producing C2 hydrocarbon compounds and aromatic compounds from methane using the non-oxidative direct conversion reactor for methane according to claim 1.
